(19) United States
(12) Patent Application Publication
(10) Pub. No.: US 2026/0306151 A1
(43) Pub. Date: Oct. 8, 2026

(54) BONE PLATE FOR TREATING FIRST TARSAL METATARSAL LISFRANC INJURIES

(71) Applicant: In2Bones, LLC, Memphis, TN (US)

(72) Inventors: Kevin E. Varner, Memphis, TN (US); Robert M. Crews, Memphis, TN (US)

(73) Assignee: In2Bones, LLC, Memphis, TN (US)

(21) Appl. No.: 18/998,588

(22) PCT Filed: Jul. 25, 2023
(86) PCT No.: PCT/US2023/028616

§ 371 Date: Jan. 27, 2025

Related U.S. Application Data
(60) Provisional application No. 63/392,923, filed on Jul. 28, 2022.

(51) Int. Cl. A61B 17/80; A61B 17/17; A61B 17/56

(52) U.S. Cl. CPC A61B 17/8061; A61B 17/1775; A61B 17/8057; A61B 2017/564

(57) ABSTRACT

An apparatus and methods are provided for a bone fusion plate for treating 1^{st} tarsal-metatarsal Lisfranc injuries. The bone fusion plate comprises an elongate member that includes a proximal contact surface for attaching to a medial cuneiform bone and a distal contact surface for attaching to a 1^{st} metatarsal bone. Proximal apertures enable implantation of proximal bone screws into the medial cuneiform bone, and distal apertures enable implantation of distal bone screws into the 1^{st} metatarsal bone. A spacing between the proximal and distal apertures positions the proximal bone screws away from a cross screw for treating a Lisfranc injury. A medial thickness of the bone fusion plate vertically implants the proximal bone screws into the greatest thickness of the medial cuneiform. The bone fusion plate includes a tapered distal tip for sliding under soft tissue, thereby facilitating a minimal incision length during implantation of the bone fusion plate.

### PRIORITY

This application claims the benefit of and priority to PCT Application No. PCT/US2023/028616 filed Jul. 25, 2023 and U.S. Provisional Application, entitled “Bone Plate For Treating First Tarsal Metatarsal Lisfranc Injuries,” filed on Jul. 28, 2022, and having application ser. no. 63/392,923, the entirety of said application being incorporated herein by reference.

### FIELD

Embodiments of the present disclosure generally relate to securing bones together. More specifically, embodiments of the disclosure relate to an apparatus and methods for a bone fusion plate for treating 1^{st} tarsal-metatarsal Lisfranc injuries that require open reduction with internal fixation.

### BACKGROUND

A Lisfranc injury, also known as a tarsal metatarsal (TMT) injury, is an injury of the foot in which one or more of the metatarsal bones are displaced from the tarsus. Lisfranc injuries may be caused by direct or indirect forces. For example, direct forces may include a crush injury or a direct blow, such as due to motor vehicle accidents or industrial injuries. These may be combined with soft-tissue injury and present as open fractures. Injuries due to indirect forces are more common, however, and often result from an axial load to a plantarflexed foot. Indirect Lisfranc injuries may occur during sports or stepping down from a stair or sidewalk.

Treatment options generally include operative or non-operative treatments. In some instances, for example, if a dislocation is less than 2 mm, the fracture may be managed with casting for six weeks. In a majority of Lisfranc injuries, however, early anatomical reduction and stable fixation is required. In some instances, closed reduction and K-wire stabilization or open reduction and stabilization—generally using screws to avoid the complication of K-wires and maintain a stable reduction—are desired treatments. Open reduction with internal fixation (ORIF) and temporary screw or K-wire fixation is the treatment of choice for severe Lisfranc injuries. Depending on the specific injury, the K-wires are typically removed after six weeks while the screws are often removed after 12 weeks.

Unlike other areas of the body having “essential” joints, the joints in the midfoot area are not “essential” and thus motion may be sacrificed to obtain stability and function. As such, the 1^{st}, 2^{nd}, and 3^{rd} TMT joints may be fused for acute purely ligamentous injuries. However, the 4^{th} and 5^{th} TMT joints typically are stabilized with K-wires as some retained motion in this area is helpful for better function. As will be appreciated, joint surface reconstruction may not be as critical as in other joints when treating fractures in the Lisfranc/midfoot area.

There is an ongoing need for the development of midfoot treatment capabilities such as that related to, for example, treating 1^{st} TMT Lisfranc injuries. Provided herein are embodiments and methods for a bone fusion plate for treating 1^{st} TMT Lisfranc injuries that require open reduction with internal fixation.

### SUMMARY

An apparatus and methods are provided for a bone fusion plate for treating 1st tarsal-metatarsal Lisfranc injuries. The bone fusion plate comprises an elongate member that includes a proximal contact surface for attaching to a medial cuneiform bone and a distal contact surface for attaching to a 1st metatarsal bone. Proximal apertures enable implantation of proximal bone screws into the medial cuneiform bone, and distal apertures enable implantation of distal bone screws into the 1st metatarsal bone. A spacing between the proximal and distal apertures positions the proximal bone screws away from a cross screw for treating a Lisfranc injury. A medial thickness of the bone fusion plate vertically implants the proximal bone screws into the greatest thickness of the medial cuneiform. The bone fusion plate includes a tapered distal tip for sliding under soft tissue, thereby facilitating a minimal incision length during implantation of the bone fusion plate.

In an exemplary embodiment, a bone fusion plate for fixating a 1st tarsal-metatarsal joint comprises: an elongate member including a proximal contact surface and a distal contact surface; one or more proximal apertures for implantation of proximal bone screws into a medial cuneiform bone; one or more distal apertures for implantation of distal bone screws into a 1st metatarsal bone; and a medial thickness and a lateral thickness for vertically implanting the proximal bone screws toward the greatest thickness of the medial cuneiform.

In another exemplary embodiment, the proximal contact surface is configured to contact a dorso-medial portion of the medial cuneiform bone. In another exemplary embodiment, the distal contact surface is configured to contact a cortical surface of the 1st metatarsal bone.

In another exemplary embodiment, any one or more of the one or more proximal apertures includes a countersink for receiving the heads of the proximal bone screws below the surface of the bone fusion plate so as to avoid potential tissue irritation. In another exemplary embodiment, any one or more of the one or more distal apertures includes a countersink for receiving the heads of the distal bone screws below the surface of the bone fusion plate so as to avoid potential tissue irritation.

In another exemplary embodiment, a spacing is disposed between the one or more proximal apertures and the one or more distal apertures. In another exemplary embodiment, the spacing is adapted to position the proximal bone screws away from a cross screw that may be used to treat a Lisfranc injury.

In another exemplary embodiment, the proximal contact surface and the distal contact surface share an intervening longitudinal angle with respect to one another. In another exemplary embodiment, the longitudinal angle is configured to cause the bone fusion plate to advantageously match cortical surface anatomies of the medial cuneiform and 1st metatarsal bones across the 1st TMT joint.

In another exemplary embodiment, the elongate member includes a tapered distal tip that is configured to slide under soft tissue during implantation of the bone fusion plate across the 1st TMT joint. In another exemplary embodiment, the tapered distal tip is configured to facilitate using a minimal incision length during implantation of the bone fusion plate. In another exemplary embodiment, the medial thickness is configured to hide the heads of proximal bone screws so as to avoid any potential tissue irritation that might otherwise occur.

In another exemplary embodiment, medial thickness and the lateral thickness are configured to direct the proximal bone screws toward the greatest thickness of the medial cuneiform bone. In another exemplary embodiment, the medial thickness is greater than the lateral thickness, such that the one or more proximal apertures are disposed at a lateral angle with respect to the proximal contact surface. In another exemplary embodiment, the lateral angle is configured to direct the proximal bone screws toward an apex of the medial cuneiform bone.

In an exemplary embodiment, a method for a bone fusion plate comprises: forming an elongate member having a proximal contact surface and a distal contact surface; adapting the proximal contact surface to be attached to a medial cuneiform bone; configuring the distal contact surface to be attached to a 1st metatarsal bone; and forming a medial thickness that orients proximal bone screws toward the greatest thickness of the medial cuneiform bone.

In another exemplary embodiment, adapting includes forming one or more proximal apertures for implantation of proximal bone screws into the medial cuneiform bone. In another exemplary embodiment, configuring includes forming one or more distal apertures for implantation of distal bone screws into the 1st metatarsal bone. In another exemplary embodiment, configuring further includes creating a spacing that provides clearance for a cross screw that may be used to treat a Lisfranc injury.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates an exemplary-use environment wherein an exemplary embodiment of a bone fusion plate is used to fixate a 1^{st} TMT joint between a medial cuneiform bone and a 1^{st} metatarsal bone;

FIG. 2 illustrates a top view of an exemplary-use environment wherein an exemplary embodiment of a bone fusion plate is used to fixate the 1^{st} TMT joint in cooperation with a cross screw that treats a Lisfranc injury;

FIG. 3 illustrates a front cross-sectional view of an exemplary-use environment wherein an exemplary embodiment of a bone fusion plate is used to orient a bone screw into an aspect of the medial cuneiform bone having the greatest quantity of usable bone;

FIG. 4 illustrates a perspective view of an exemplary embodiment of a bone fusion plate configured for treating 1^{st} TMT Lisfranc injuries, according to the present disclosure;

FIG. 5 illustrates a cross-sectional view of the bone fusion plate of FIG. 4, taken along a midline; and

FIG. 6 illustrates a cross-sectional view of the bone fusion plate of FIG. 5, taken a long line 6-6.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the bone fusion plate and methods disclosed herein may be practiced without these specific details. In other instances, specific numeric references such as “first bone screw,” may be made. However, the specific numeric reference should not be interpreted as a literal sequential order but rather interpreted that the “first bone screw” is different than a “second bone screw.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Lisfranc injuries and 1^{st} tarsal metatarsal (TMT) injuries may be caused by direct or indirect forces, and treatment options typically include operative or non-operative treatments. Open reduction with internal fixation (ORIF) and temporary screw or K-wire fixation is the treatment of choice for severe 1^{st} TMT Lisfranc injuries. There is an ongoing need for the development of midfoot treatment capabilities such as that related to, for example, treating 1^{st} TMT Lisfranc injuries. Provided herein are embodiments and methods for a bone fusion plate configured to facilitate treating 1^{st} TMT Lisfranc injuries that require open reduction with internal fixation.

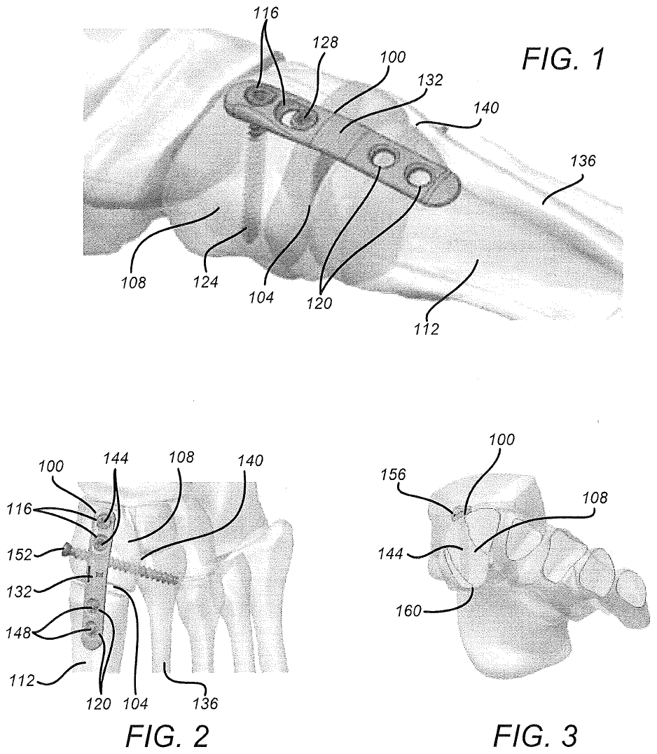

FIG. 1 illustrates an exemplary-use environment wherein an exemplary embodiment of a bone fusion plate 100 is used to fixate a 1^{st} TMT joint 104 between a medial cuneiform bone 108 and a 1^{st} metatarsal bone 112 in accordance with the present disclosure. The bone fusion plate 100 is a generally elongate member adapted to be implanted onto the dorso-medial portion of the medial cuneiform bone 108. The bone fusion plate 100 includes one or more proximal apertures 116 and one or more distal apertures 120 that are configured to receive bone screws for fixating the 1^{st} TMT joint 104.

In the exemplary-use environment of FIG. 1, a vertical bone screw 124 is implanted through a first proximal aperture 116 into an aspect of the medial cuneiform bone 108 having the greatest quantity of usable bone. An angled bone screw 128 is shown implanted through a second proximal aperture 116 into the medial cuneiform bone 108, however it is contemplated that a vertical bone screw may be implanted through the second proximal aperture 116, without limitation. Bone screws are not shown disposed in the distal apertures 120 for the sake of clarity of illustration. The distal apertures 120 are disposed in locations of the bone fusion plate 100 that are suitable for receiving bone screws to be implanted into the 1^{st} metatarsal bone 112. As discussed herein, the bone fusion plate 100 includes a spacing 132 between the proximal and distal apertures 116, 120 that is configured to accommodate a cross screw for fixating a Lisfranc joint 140 between the medial cuneiform bone 108 and a 2^{nd} metatarsal bone 136, as shown in FIG. 2.

FIG. 2 illustrates a top view of an exemplary-use environment wherein an exemplary embodiment of a bone fusion plate 100 is used to fixate the 1^{st} TMT joint 104 in accordance with the present disclosure. Proximal bone screws 144 are implanted through the proximal apertures 116 and attach the bone fusion plate 100 to the medial cuneiform bone 108. One or more of the proximal bone screws 144 preferably are vertically implanted into an aspect of the medial cuneiform bone 108 having the greatest quantity of usable bone, as described herein. Distal bone screws 148 are implanted through the distal apertures 120 and attach the bone fusion plate 100 to the 1^{st} metatarsal bone 112. As will be appreciated, the distal bone screws 148 may be implanted at an angle that is normal to the cortical surface of the 1^{st} metatarsal bone 112. Further, as shown in FIG. 2, the spacing 132 between the proximal and distal apertures 116, 120 provides clearance for a cross screw 152 to be implanted across the medial cuneiform bone 108 and into the 2^{nd} metatarsal bone 136 to fixate the Lisfranc joint 140. Thus, the bone fusion plate 100 is configured to fixate the 1^{st} TMT joint 104 while cooperating with treatments for a Lisfranc injury.

FIG. 3 illustrates a front cross-sectional view of an exemplary-use environment wherein an exemplary embodiment of a bone fusion plate 100 is used to advantageously orient a proximal bone screw 144 within a medial cuneiform bone 108 in accordance with the present disclosure. As shown in FIG. 3, the bone fusion plate 100 is configured to orient the proximal bone screw 144 into an aspect of the medial cuneiform bone 108 having the greatest quantity of usable bone. Unlike conventional bone plates that direct bones screw at an angle normal to the cortical surface, the bone fusion plate 100 includes a medial thickness 156 configured to direct the proximal bone screw 144 toward an apex 160 of a triangular cross-sectional shape of the medial cuneiform bone 108. It is contemplated that the added material comprising the medial thickness 156 of the bone fusion plate 100 further serves to hide heads of the proximal bone screws 144 so as to avoid any potential tissue irritation that might otherwise occur.

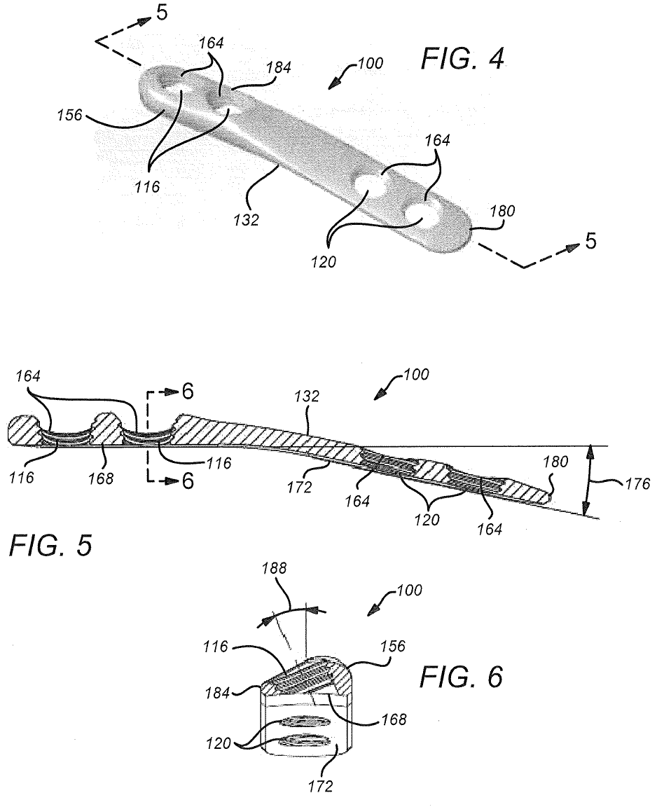

FIGS. 4-6 illustrate an exemplary embodiment of a bone fusion plate 100 configured for fixating the 1^{st} TMT joint 104 between the medial cuneiform bone 108 and the 1^{st} metatarsal bone 112 to treat 1^{st} TMT Lisfranc injuries, as described herein. In general, the bone fusion plate 100 is an elongate member adapted to be attached to the dorso-medial portion of the medial cuneiform bone 108. The bone fusion plate 100 includes one or more proximal apertures 116 and one or more distal apertures 120 that are configured to receive bone screws for fixating the 1^{st} TMT joint 104. The proximal and distal apertures 116, 120 may each include a countersink 164 configured to receive the heads of the bone screws below the surface of the bone fusion plate so as to avoid any potential tissue irritation that might otherwise occur. The bone fusion plate 100 further includes a spacing 132 disposed between the proximal and distal apertures 116, 120. The spacing 132 is adapted to position bone screws implanted in the proximal apertures 116 away from a cross screw 152 (see FIG. 2) that may be used to treat a Lisfranc injury.

As best shown in FIG. 5, the bone fusion plate 100 includes a proximal contact surface 168 that is configured to be placed into contact with the cortical surface of the medial cuneiform bone 108. A distal contact surface 172 comprising the bone fusion plate 100 is configured to be placed into contact with the cortical surface of the 1^{st} metatarsal bone 112. As shown in FIG. 5, the proximal contact surface 168 and the distal contact surface 172 share an intervening longitudinal angle 176 with respect to one another. The longitudinal angle 176 causes the bone fusion plate 100 to advantageously bridge the 1^{st} TMT joint 104 and match the cortical surface anatomies of the medial cuneiform and 1^{st} metatarsal bones 108, 112. In the illustrated embodiment of FIGS. 4-6, wherein the bone fusion plate 100 is configured to treat the 1^{st} TMT joint 104, the longitudinal angle 176 may be about 12 degrees. In some embodiments, however, wherein the bone fusion plate 100 is adapted to treat joints other than the 1^{st} TMT joint 104, the longitudinal angle 176 may be more than or less than 12 degrees, without limitation.

As shown in FIG. 5, the bone fusion plate 100 includes a tapered distal tip 180. The tapered distal tip 180 is configured to slide under soft tissue during implantation of the bone fusion plate 100 across the 1^{st} TMT joint 104. It is contemplated that the tapered distal tip 180 facilitates using a minimal incision length during implantation of the bone fusion plate 100. It is further contemplated that the tapered distal tip 180 may be altered, as may be found to be beneficial in practice, without limitation.

As described hereinabove in connection with FIG. 3, the bone fusion plate 100 is configured to orient the proximal bone screw 144 into an aspect of the medial cuneiform bone 108 having the greatest quantity of usable bone. In the embodiment shown in FIGS. 4-6, the bone fusion plate 100 includes a medial thickness 156 and a lateral thickness 184 that are configured to direct a proximal bone screw 144 toward an apex 160 of the triangular cross-sectional shape of the medial cuneiform bone 108 (see FIG. 3). As best shown in FIG. 6, the medial thickness 156 is greater than the lateral thickness 184, such that the proximal apertures 116 are disposed at a lateral angle 188 with respect to the proximal contact surface 168. It should be borne in mind that the lateral angle 188 is configured to direct a bone screw toward the apex 160 of the medial cuneiform bone 108, as best shown in FIG. 3. In the illustrated embodiment of FIGS. 4-6, wherein the bone fusion plate 100 is configured to treat the 1^{st} TMT joint 104, the lateral angle 188 may be about 25 degrees. In some embodiments, however, wherein the bone fusion plate 100 may be adapted to treat joints other than the 1^{st} TMT joint 104, the lateral angle 188 may be greater than or less than 25 degrees, without limitation. Further, it should be understood that the medial thickness 156 of the bone fusion plate 100 further serves to hide heads of bone screws so as to avoid any potential tissue irritation that might otherwise occur.

While the bone fusion plate and methods have been described in terms of particular variations and illustrative figures, those of ordinary skill in the art will recognize that the bone fusion plate is not limited to the variations or figures described. In addition, where methods and steps described above indicate certain events occurring in certain order, those of ordinary skill in the art will recognize that the ordering of certain steps may be modified and that such modifications are in accordance with the variations of the bone fusion plate. Additionally, certain of the steps may be performed concurrently in a parallel process, when possible, as well as performed sequentially as described above. To the extent there are variations of the bone fusion plate, which are within the spirit of the disclosure or equivalent to the bone fusion plate found in the claims, it is the intent that this patent will cover those variations as well. Therefore, the present disclosure is to be understood as not limited by the specific embodiments described herein, but only by scope of the appended claims.

What is claimed is:

1. A bone fusion plate for fixating a 1^{st} tarsal-metatarsal joint, comprising:
an elongate member including a proximal contact surface and a distal contact surface;
one or more proximal apertures for implantation of proximal bone screws into a medial cuneiform bone;
one or more distal apertures for implantation of distal bone screws into a 1^{st} metatarsal bone; and
a medial thickness and a lateral thickness for vertically implanting the proximal bone screws toward the greatest thickness of the medial cuneiform.

2. The bone fusion plate of claim 1, wherein the proximal contact surface is configured to contact a dorso-medial portion of the medial cuneiform bone.

3. The bone fusion plate of claim 1, wherein the distal contact surface is configured to contact a cortical surface of the 1^{st} metatarsal bone.

4. The bone fusion plate of claim 1, wherein any one or more of the one or more proximal apertures includes a countersink for receiving the heads of the proximal bone screws below the surface of the bone fusion plate so as to avoid potential tissue irritation.

5. The bone fusion plate of claim 1, wherein any one or more of the one or more distal apertures includes a countersink for receiving the heads of the distal bone screws below the surface of the bone fusion plate so as to avoid potential tissue irritation.

6. The bone fusion plate of claim 1, wherein a spacing is disposed between the one or more proximal apertures and the one or more distal apertures.

7. The bone fusion plate of claim 6, wherein the spacing is adapted to position the proximal bone screws away from a cross screw that may be used to treat a Lisfranc injury.

8. The bone fusion plate of claim 1, wherein the proximal contact surface and the distal contact surface share an intervening longitudinal angle with respect to one another.

9. The bone fusion plate of claim 8, wherein the longitudinal angle is configured to cause the bone fusion plate to advantageously match cortical surface anatomies of the medial cuneiform and 1^{st} metatarsal bones across the 1^{st} TMT joint.

10. The bone fusion plate of claim 1, wherein the elongate member includes a tapered distal tip that is configured to slide under soft tissue during implantation of the bone fusion plate across the 1^{st} TMT joint.

11. The bone fusion plate of claim 10, wherein the tapered distal tip is configured to facilitate using a minimal incision length during implantation of the bone fusion plate.

12. The bone fusion plate of claim 1, wherein the medial thickness is configured to hide the heads of proximal bone screws so as to avoid any potential tissue irritation that might otherwise occur.

13. The bone fusion plate of claim 1, wherein medial thickness and the lateral thickness are configured to direct the proximal bone screws toward the greatest thickness of the medial cuneiform bone.

14. The bone fusion plate of claim 13, wherein the medial thickness is greater than the lateral thickness, such that the one or more proximal apertures are disposed at a lateral angle with respect to the proximal contact surface.

15. The bone fusion plate of claim 14, wherein the lateral angle is configured to direct the proximal bone screws toward an apex of the medial cuneiform bone.

16. A method for a bone fusion plate, comprising:
forming an elongate member having a proximal contact surface and a distal contact surface;
adapting the proximal contact surface to be attached to a medial cuneiform bone;
configuring the distal contact surface to be attached to a 1^{st} metatarsal bone; and
forming a medial thickness that orients proximal bone screws toward the greatest thickness of the medial cuneiform bone.

17. The method of claim 16, wherein adapting includes forming one or more proximal apertures for implantation of proximal bone screws into the medial cuneiform bone.

18. The method of claim 17, wherein configuring includes forming one or more distal apertures for implantation of distal bone screws into the 1^{st} metatarsal bone.

19. The method of claim 18, wherein configuring further includes creating a spacing that provides clearance for a cross screw that may be used to treat a Lisfranc injury.
